(19) United States
(12) Patent Application Publication
(10) Pub. No.: US 2026/0301879 A1
(43) Pub. Date: Oct. 1, 2026

(54) METHODS FOR PROVIDING POLYMER MEMBRANES FOR SOLVENT SEPARATION

(71) Applicant: Georgia Tech Research Corporation, Atlanta, GA (US)

(72) Inventors: Rampi Ramprasad, Atlanta, GA (US); Janhavi Nistane, Atlanta, GA (US); Harikrishna Sahu, Atlanta, GA (US); Ryan Lively, Atlanta, GA (US)

(21) Appl. No.: 19/633,536

(22) Filed: Mar. 30, 2026

Related U.S. Application Data
(60) Provisional application No. 63/779,437, filed on Mar. 28, 2025.

(51) Int. Cl. G16C 20/20; G06N 3/04; G06N 3/08; G16C 20/30; G16C 20/70; G16C 20/90

(52) U.S. Cl. CPC G16C 20/20; G06N 3/04; G06N 3/08; G16C 20/30; G16C 20/70; G16C 20/90

(57) ABSTRACT

A method for identifying polymer membranes for solvent separation includes receiving experimental diffusivity data comprising measurements of solvent diffusivity in polymers and receiving simulated diffusivity data comprising computationally generated solvent diffusivity values for polymer-solvent systems. The method includes training a multi-task machine learning model using both data types, wherein the model learns correlations between experimental and simulated data. The method includes encoding a physics-based relationship into the model, constraining predictions according to a physical law governing solvent transport. The method includes generating diffusivity predictions for polymer candidates, calculating permeability and permselectivity values based on the predictions, and identifying polymer membrane candidates based on the calculated values.

### CROSS-REFERENCE TO RELATED APPLICATIONS

This application claims priority to U.S. Provisional Application No. 63/779,437, filed 28 Mar. 2025, which is hereby incorporated by reference in its entirety.

### FIELD OF INVENTION

The present disclosure relates to polymer membrane design for solvent separation applications, and more particularly to machine learning methods that integrate experimental and simulated diffusivity data through multi-task and physics-enforced learning to identify polymer membranes with optimized permeability and selectivity for binary organic solvent separations.

### BACKGROUND

Separating organic solvents is a fundamental process in the chemical industry for producing fuels, chemicals, and other derived products. A notable example is the separation of aromatic compounds, such as toluene, from aliphatic compounds like n-heptane, which is relevant for producing cleaner motor fuels with reduced aromatic content. Traditional separation methods such as extractive distillation or liquid-liquid extraction face challenges in separating such mixtures due to the close boiling points and similar physiochemical properties of the solvent components. Pervaporation presents an alternative approach by utilizing differences in the permeation rates of organic solvent molecules across a membrane for separation rather than relative volatility alone. Pervaporation-based approaches offer advantages such as enhanced safety, cost-efficiency, and reduced energy consumption compared to distillation-based methods. In such separations, a liquid phase solvent mixture is introduced to one side of a polymer membrane, while the permeate exits from the opposite side in a vapor phase. Polymeric membranes are widely used for such separations due to their low cost of fabrication and ease of scaling up.

For solvent separations, membrane properties such as solvent permeability and permselectivity are relevant parameters. The mass transport in the pervaporation process follows the solution-diffusion mechanism, which states that the permeability through a dense membrane is the product of diffusivity and solubility coefficient. Knowledge of pure component diffusivity and sorption isotherms is relevant for predicting membrane separation performance for complex mixtures. Experimental data for solvent diffusivity, typically obtained through gravimetric sorption or timelag measurements, is limited and resource-intensive to expand. Classical molecular dynamics simulations, while effective for calculating diffusivity, are constrained by computational costs and the need for accurate force-field parameters. Machine learning-driven methods offer a route for diffusivity predictions while addressing the resource and scalability limitations of conventional experimental and computational techniques.

However, current machine learning models for diffusivity encounter difficulties in extrapolating beyond the polymer-property space encompassed by their training data. This limitation reflects a challenge of machine learning models in reliably predicting outcomes outside the training set. Additionally, identifying membranes that possess both high solvent permeability and permselectivity remains challenging due to the inherent trade-off between these properties. Furthermore, there is a desire to identify polymer membranes that are environmentally friendly and potentially recyclable, as some existing high-performance membranes contain halogenated compounds that raise environmental concerns.

What is needed, therefore, is an improved approach for predicting solvent diffusivity in polymers that can generalize beyond training data domains, and methods for identifying polymer membrane candidates that achieve favorable permeability and selectivity characteristics while also considering environmental sustainability factors.

### SUMMARY

This summary is provided to introduce a selection of concepts in a simplified form that are further described below in the detailed description. This summary is not intended to identify key features or essential features of the claimed subject matter, nor is it intended to be used as an aid in determining the scope of the claimed subject matter.

A method for identifying polymer membranes for solvent separation can include receiving experimental diffusivity data including measurements of solvent diffusivity in polymers. The method can include receiving simulated diffusivity data including computationally generated solvent diffusivity values for polymer-solvent systems. The method can include training a multi-task machine learning model using both the experimental diffusivity data and the simulated diffusivity data, wherein the multi-task machine learning model learns correlations between the experimental diffusivity data and the simulated diffusivity data. The method can include encoding a physics-based relationship into the multi-task machine learning model, wherein the physics-based relationship constrains predictions of the multi-task machine learning model according to a physical law governing solvent transport. The method can include generating, using the trained multi-task machine learning model, diffusivity predictions for a plurality of polymer candidates. The method can include calculating permeability values and permselectivity values for the plurality of polymer candidates based on the diffusivity predictions. The method can include identifying one or more polymer membrane candidates from the plurality of polymer candidates based on the calculated permeability values and permselectivity values.

A system for polymer membrane design for solvent separation can include a processor. The system can include a memory storing instructions that, when executed by the processor, cause the processor to receive a combined dataset including experimental diffusivity data and simulated diffusivity data for polymer-solvent systems. The instructions can cause the processor to train a multi-task machine learning model on the combined dataset, wherein the multi-task machine learning model includes a neural network architecture configured to learn from both the experimental diffusivity data and the simulated diffusivity data. The instructions can cause the processor to incorporate a physics-enforced constraint into the multi-task machine learning model, wherein the physics-enforced constraint encodes a relationship between solvent diffusivity and at least one of solvent molar volume or temperature. The instructions can cause the processor to generate diffusivity predictions for a set of polymer candidates using the trained multi-task machine learning model. The instructions can cause the processor to compute permeability and ideal permselectivity for each polymer candidate in the set based on the diffusivity predictions. The instructions can cause the processor to output polymer membrane candidates having permeability and ideal permselectivity values exceeding predetermined thresholds.

A non-transitory computer-readable medium can store instructions that, when executed by a processor, cause the processor to perform operations including combining experimental diffusivity data with simulated diffusivity data to form a training dataset for polymer-solvent systems. The operations can include training a physics-enforced neural network model using the training dataset, wherein the physics-enforced neural network model incorporates an Arrhenius-based temperature dependence relationship for solvent diffusivity. The operations can include predicting solvent diffusivity values for a plurality of polymers using the trained physics-enforced neural network model. The operations can include determining permeability and permselectivity metrics for the plurality of polymers based on the predicted solvent diffusivity values. The operations can include identifying non-halogenated polymer candidates from the plurality of polymers that satisfy target permeability and permselectivity criteria for a binary solvent separation application.

The foregoing general description of the illustrative embodiments and the following detailed description thereof are merely exemplary aspects of the teachings of this disclosure and are not restrictive.

### BRIEF DESCRIPTION OF FIGURES

Non-limiting and non-exhaustive examples are described with reference to the following figures.

FIG. 1 is a block diagram of an example system that may be used to provide polymer membrane design for solvent separation, according to examples of the disclosed technology.

FIG. 2 is a flow diagram for a method for generating simulated diffusivity data for polymer-solvent systems, according to examples of the disclosed technology.

FIG. 3 illustrates a Venn diagram depicting expansion of a diffusivity dataset through machine learning conversion, according to examples of the disclosed technology.

FIG. 4 is a block diagram of an example system that may be used to provide machine learning model training and prediction for solvent diffusivity, according to examples of the disclosed technology.

FIG. 5A illustrates a sorption uptake machine learning model system, according to examples of the disclosed technology.

FIG. 5B illustrates a diffusivity machine learning model system, according to examples of the disclosed technology.

FIG. 6 illustrates a trade-off plot for polymer membrane candidates in solvent separation applications, according to examples of the disclosed technology.

FIG. 7A illustrates a standard neural network architecture for predicting solvent diffusivity, according to examples of the disclosed technology.

FIG. 7B illustrates a physics-enforced neural network architecture incorporating an empirical solvent volume law, according to examples of the disclosed technology.

FIG. 7C illustrates a physics-enforced neural network architecture based on Arrhenius temperature dependence, according to examples of the disclosed technology.

### DETAILED DESCRIPTION

The following description sets forth exemplary aspects of the present disclosure. It should be recognized, however, that such description is not intended as a limitation on the scope of the present disclosure. Rather, the description also encompasses combinations and modifications to those exemplary aspects described herein.

A detailed description of systems, devices, and methods consistent with embodiments of the present disclosure is provided below. While several embodiments are described, it should be understood that disclosure is not limited to any one embodiment, but instead encompasses numerous alternatives, modifications, and equivalents. In addition, while numerous specific details are set forth in the following description in order to provide a thorough understanding of the embodiments disclosed herein, some embodiments can be practiced without some or all of these details. Moreover, for the purpose of clarity, certain technical material that is known in the related art has not been described in detail in order to avoid unnecessarily obscuring the disclosure.

The present disclosure relates to systems and methods for polymer membrane design for solvent separation. Separating organic solvents represents a process in the chemical industry for producing fuels, chemicals, and other derived products. A binary solvent separation application can comprise toluene-heptane separation for producing cleaner motor fuels with reduced aromatic content. Traditional separation methods such as extractive distillation or liquid-liquid extraction face challenges in separating such mixtures due to close boiling points and similar physiochemical properties of the solvent components. Pervaporation presents an alternative approach by utilizing differences in permeation rates of organic solvent molecules across a membrane for separation rather than relative volatility alone.

A method for identifying polymer membranes for solvent separation can leverage machine learning techniques that integrate experimental and simulated diffusivity data. Experimental data provides accurate ground truth measurements but remains limited in scope and grows slowly over time. Simulated data generated through computational methods can explore chemical spaces beyond the reach of experimental datasets, though such simulated data exhibits lower accuracy compared to experimental measurements. By combining these data sources through multi-task learning approaches, machine learning models can achieve improved generalizability when predicting solvent diffusivity in polymers.

A system for polymer membrane design for solvent separation can incorporate physics-enforced learning to further enhance predictive performance. Physics-enforced neural networks encode physical laws governing solvent transport into the model architecture, enabling more accurate predictions when extrapolating beyond training data. Such physical laws can include relationships between solvent molar volume and diffusivity, as well as Arrhenius-based temperature dependence relationships. The integration of physics-based constraints with multi-task learning enables the identification of polymer membranes with optimized permeability and selectivity for binary organic solvent separations.

Polymer membranes for solvent separation can be characterized by permeability and permselectivity properties. Permeability represents the flux normalized by membrane thickness and driving force, while permselectivity represents the ratio of permeabilities for different solvent components. According to the solution-diffusion mechanism, permeability through a dense membrane equals the product of diffusivity and solubility coefficient. Machine learning models trained on combined experimental and simulated data can predict these properties for large numbers of polymer candidates, enabling identification of polymer membranes that achieve both high permeability and high permselectivity for target solvent separations.

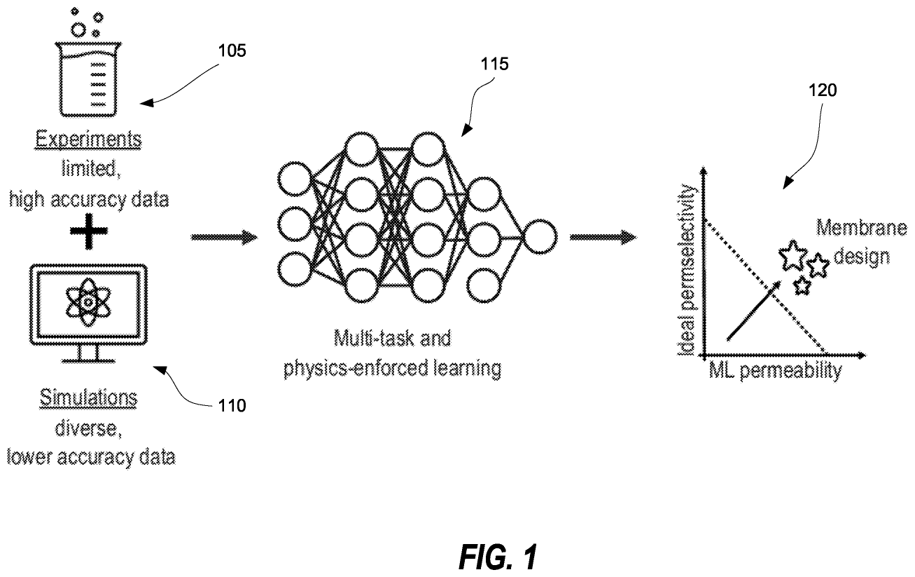

Referring to FIG. 1, a block diagram illustrates a machine learning system for polymer membrane design for solvent separation. The system receives experimental data 105 and simulated data 110 as inputs for training a multi-task and physics-enforced learning model 115. The multi-task and physics-enforced learning model 115 processes the combined inputs to generate a membrane design output 120.

The experimental data 105 can comprise measurements of solvent diffusivity in polymers. The experimental data 105 can be characterized as limited but high accuracy data. In some cases, the experimental data 105 comprises an experimental diffusivity dataset of 2045 polymer-solvent systems. In other cases, the experimental data 105 comprises 2421 experimental systems when both activity-dependent and concentration-dependent experimental diffusivity data are included. The experimental data 105 can be referred to herein as experimental diffusivity data.

The simulated data 110 can comprise computationally generated solvent diffusivity values for polymer-solvent systems. The simulated data 110 can be characterized as diverse but lower accuracy data compared to the experimental data 105. In some cases, the simulated data 110 comprises a simulated diffusivity dataset of 623 systems including 91 polymers and 69 solvents. The simulated data 110 can be referred to herein as simulated diffusivity data.

With continued reference to FIG. 1, the multi-task and physics-enforced learning model 115 receives both the experimental data 105 and the simulated data 110 as inputs. The multi-task and physics-enforced learning model 115 can comprise a neural network architecture configured to learn from both the experimental data 105 and the simulated data 110. The multi-task and physics-enforced learning model 115 learns correlations between the experimental data 105 and the simulated data 110. The multi-task and physics-enforced learning model 115 can be referred to herein as a multi-task machine learning model. A combined dataset comprising the experimental data 105 and the simulated data 110 can comprise 3044 systems including 154 polymers and 176 solvents.

The system can include a processor and a memory storing instructions that, when executed by the processor, cause the processor to receive the combined dataset comprising the experimental data 105 and the simulated data 110 for polymer-solvent systems. The instructions can further cause the processor to train the multi-task and physics-enforced learning model 115 on the combined dataset.

As further shown in FIG. 1, the membrane design output 120 comprises a graph plotting ideal permselectivity on a vertical axis against ML permeability on a horizontal axis. The membrane design output 120 includes a solid line and a dashed line representing performance boundaries. Star markers in the membrane design output 120 indicate polymer membrane candidates in an upper right region of the plot where both permeability and ideal permselectivity values are elevated. The system identifies polymer membrane candidates from the upper right region of the membrane design output 120 based on calculated permeability values and permselectivity values derived from diffusivity predictions generated by the multi-task and physics-enforced learning model 115.

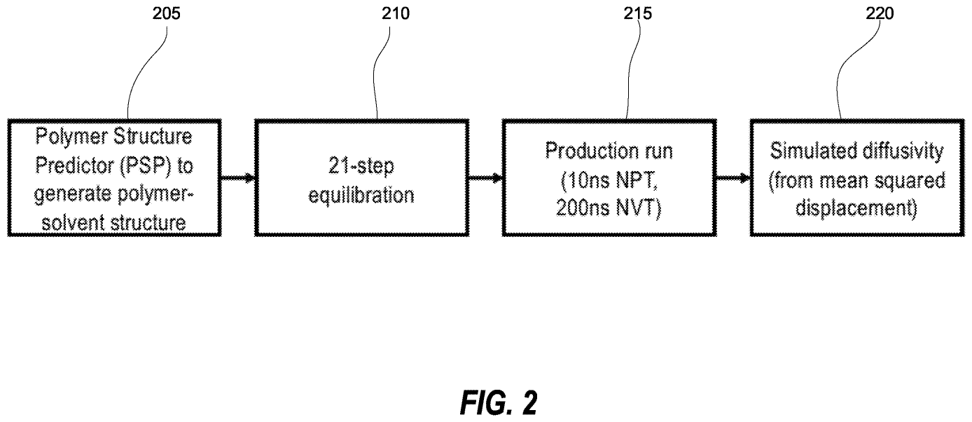

Referring to FIG. 2, a flowchart illustrates a simulation protocol 200 used to calculate the diffusivity of solvents within polymers. The simulation protocol 200 comprises a sequence of steps for generating the simulated data 110 described with reference to FIG. 1. The simulated data 110 can be generated using molecular dynamics simulations that calculate diffusivity values from mean squared displacement analysis of polymer-solvent structures.

The simulation protocol 200 begins with a step 205 where a Polymer Structure Predictor (PSP) generates polymer-solvent structures. The Polymer Structure Predictor can comprise an open-source tool for generating polymer and solvent structures for molecular dynamics simulations. The step 205 can be referred to herein as generating polymer-solvent structures using a polymer structure predictor. The polymer-solvent structures generated in the step 205 can consist of approximately 150 atoms per polymer chain, with the entire system totaling 4000-5000 atoms. The simulations can operate within a dilute solvent concentration regime for diffusivity calculations.

With continued reference to FIG. 2, the simulation protocol 200 proceeds to a step 210 involving an equilibration procedure on the polymer-solvent structures. The step 210 comprises a 21-step equilibration process to ensure that the polymers are properly equilibrated within the simulation environment. The equilibration procedure can be referred to herein as performing a multi-step equilibration procedure on the polymer-solvent structures. Following the 21-step equilibration process, the polymer-solvent systems can undergo an additional 10 ns equilibration in the NPT ensemble. The molecular dynamics simulations can use GAFF2-Gasteiger as the force field for generating the simulated data 110.

The simulation protocol 200 continues to a step 215 comprising a production run in NPT and NVT ensembles. The step 215 can comprise executing a production run comprising an NPT ensemble phase followed by an NVT ensemble phase. In some cases, the production run comprises 10 nanoseconds in the NPT ensemble followed by 200 nanoseconds in the NVT ensemble. The molecular dynamics simulations can use a Nosé-Hoover thermostat and barostat with a damping parameter of 100 time steps each. The molecular dynamics simulations can use a time step of 1 fs for all simulations.

As further shown in FIG. 2, the simulation protocol 200 concludes with a step 220 where simulated diffusivity is calculated from mean squared displacement analysis. The step 220 can be referred to herein as calculating diffusivity values from mean squared displacement analysis. The diffusion coefficient D can be calculated according to the following equation:

where N represents the number of solvent molecules, t represents the simulation time, rᵢ(t) represents the position vector of the solvent at time t, and rᵢ(0) represents the position vector at the initial time 0.

The diffusivity values calculated in the step 220 can be verified to be in the Fickian regime by confirming that the slope of the log displacement-log time plot remains within the range of 0.95-1.05. If the slope value falls outside this range, the diffusivity runs can be excluded from subsequent calculations. The standard block average method can be used to estimate simulated uncertainty from 10 blocks for diffusivity calculations. The simulated diffusivity data generated through the simulation protocol 200 can be combined with the experimental data 105 for training the multi-task and physics-enforced learning model 115 as described with reference to FIG. 1.

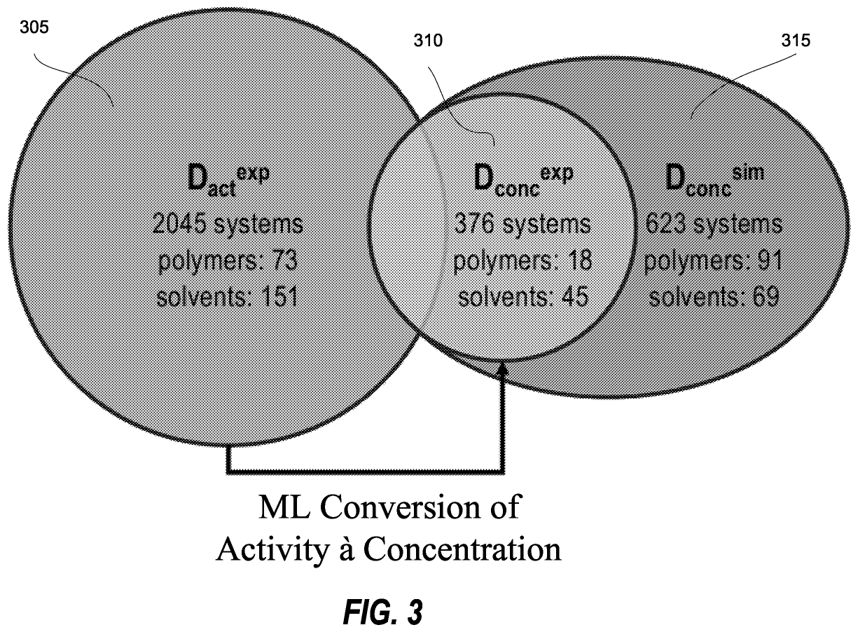

Referring to FIG. 3, a Venn diagram illustrates the expansion of a diffusivity dataset through machine learning conversion of activity data to concentration data. The diagram shows three overlapping circles representing different data sources used in the multi-task learning approach for training the multi-task and physics-enforced learning model 115 described with reference to FIG. 1.

An experimental activity dataset 305 is shown on the left side of the Venn diagram. The experimental activity dataset 305 contains 2045 systems with 73 polymers and 151 solvents. The experimental activity dataset 305 comprises activity-dependent diffusivity measurements where solvent activity serves as the concentration variable. The experimental activity dataset 305 can be referred to herein as experimental activity-dependent diffusivity data.

With continued reference to FIG. 3, an experimental concentration dataset 310 is shown in the center of the Venn diagram. The experimental concentration dataset 310 contains 376 systems with 18 polymers and 45 solvents. The experimental concentration dataset 310 comprises concentration-dependent diffusivity measurements where solvent concentration serves as the concentration variable. The experimental activity dataset 305 and the experimental concentration dataset 310 partially overlap, indicating shared data between these two experimental sources. The experimental concentration dataset 310 can be referred to herein as concentration-dependent diffusivity data.

A simulated concentration dataset 315 is shown on the right side of the Venn diagram. The simulated concentration dataset 315 contains 623 systems with 91 polymers and 69 solvents. The simulated concentration dataset 315 comprises computationally generated concentration-dependent diffusivity values generated through the simulation protocol 200 described with reference to FIG. 2. The experimental concentration dataset 310 and the simulated concentration dataset 315 partially overlap, indicating shared data between the experimental and simulated concentration-dependent datasets.

As further shown in FIG. 3, a bracket beneath the experimental activity dataset 305 and the experimental concentration dataset 310 indicates that machine learning conversion is applied to transform activity-dependent data to concentration-dependent format. Training the multi-task and physics-enforced learning model 115 can comprise converting experimental activity-dependent diffusivity data to concentration-dependent diffusivity data using a sorption uptake machine learning model. The sorption uptake machine learning model predicts specific uptake concentration values that enable conversion between activity-dependent and concentration-dependent formats.

The converted concentration-dependent diffusivity data can be combined with the simulated diffusivity data for training the multi-task and physics-enforced learning model 115. Combining experimental diffusivity data with simulated diffusivity data forms a training dataset for polymer-solvent systems. The instructions stored in the memory can cause the processor to convert experimental activity-dependent diffusivity data to concentration-dependent diffusivity data using the sorption uptake machine learning model. The instructions can further cause the processor to combine the converted concentration-dependent diffusivity data with the simulated diffusivity data to form the combined dataset.

One-hot encoding can be used to differentiate between experimental and simulated data when both are present in the training dataset. The one-hot encoding enables the multi-task and physics-enforced learning model 115 to learn correlations between the experimental data 105 and the simulated data 110 while maintaining distinction between the data sources during training.

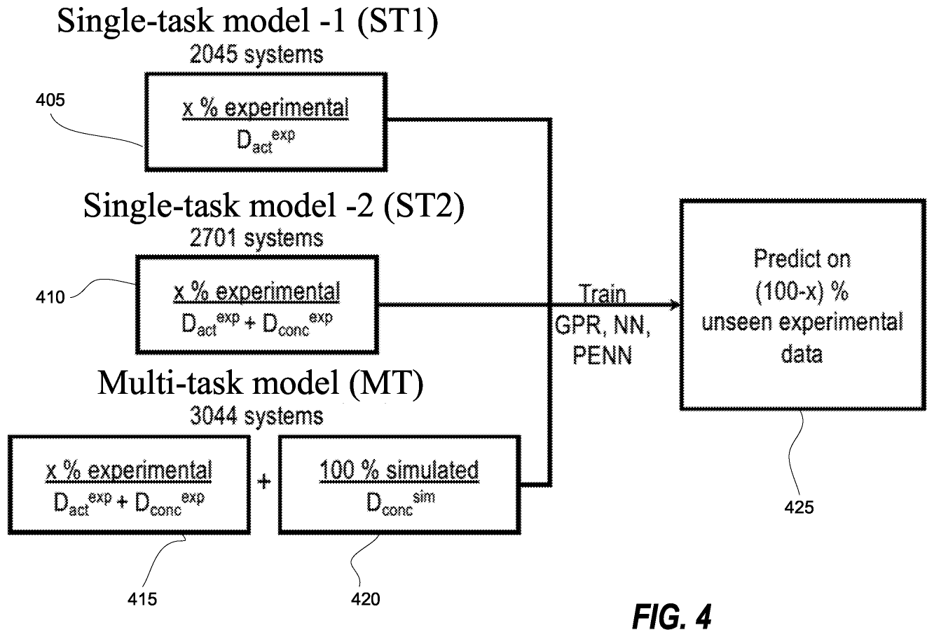

Referring to FIG. 4, a block diagram illustrates a machine learning model training and prediction system for solvent diffusivity. The system depicts three different model configurations used for training and evaluation purposes. The system enables comparison between single-task models trained solely on experimental data and multi-task models that leverage both experimental and simulated data sources to improve prediction generalizability across broader chemical spaces.

A single-task model experimental data block 405 represents a first single-task model (ST1) trained on 2045 systems using x percent experimental activity-dependent diffusivity data. The single-task model experimental data block 405 corresponds to training using the experimental activity dataset 305 described with reference to FIG. 3. The first single-task model provides a baseline for comparison against models trained on larger or more diverse datasets.

With continued reference to FIG. 4, a single-task model combined data block 410 represents a second single-task model (ST2) trained on 2701 systems using x percent experimental data comprising both activity-dependent and concentration-dependent experimental diffusivity data. The single-task model combined data block 410 incorporates data from both the experimental activity dataset 305 and the experimental concentration dataset 310 described with reference to FIG. 3. The second single-task model demonstrates the effect of combining different experimental data formats on prediction performance.

A multi-task experimental data block 415 and a multi-task simulated data block 420 together represent a multi-task model (MT) trained on 3044 systems. The multi-task experimental data block 415 contains x percent experimental data including both activity-dependent and concentration-dependent experimental diffusivity data. The multi-task simulated data block 420 contains 100 percent simulated concentration-dependent diffusivity data corresponding to the simulated concentration dataset 315 described with reference to FIG. 3. The multi-task model learns correlations between the experimental data 105 and the simulated data 110 as described with reference to FIG. 1.

As further shown in FIG. 4, the single-task model experimental data block 405, the single-task model combined data block 410, and the combination of the multi-task experimental data block 415 and the multi-task simulated data block 420 each feed into a training process. The training process utilizes Gaussian Process Regression (GPR), Neural Networks (NN), and Physics-Enforced Neural Networks (PENN). The multi-task machine learning model can use Gaussian Process Regression as an alternative algorithm to neural networks, which can be effective in data-scarce scenarios with limited training data.

The trained models generate outputs directed to a prediction output block 425. The prediction output block 425 performs predictions on (100-x) percent unseen experimental data. The prediction output block 425 generates diffusivity predictions for a plurality of polymer candidates using the trained multi-task machine learning model. The prediction output block 425 can generate diffusivity predictions for a set of polymer candidates using the trained multi-task machine learning model as described with reference to FIG. 1.

The multi-task and physics-enforced learning model 115 can incorporate a physics-enforced constraint into the multi-task machine learning model. The physics-enforced constraint encodes a relationship between solvent diffusivity and at least one of solvent molar volume or temperature. Encoding a physics-based relationship into the multi-task machine learning model constrains predictions of the multi-task machine learning model according to a physical law governing solvent transport. The physics-based relationship can comprise an empirical power law relationship between solvent molar volume and diffusivity or an Arrhenius-based temperature dependence relationship.

The polymer and solvent numerical representations can use Polymer Genome-based hierarchical fingerprinting. The hierarchical fingerprinting includes atomic-level fingerprints considering atomic triples, block-level fingerprints examining larger-scale blocks such as benzene rings, and morphological descriptors. The fingerprinting scheme can include quantitative structure-property relationship (QSPR) descriptors such as Van der Waals volume, surface area, and topological polar surface area (TPSA).

The neural network model can use a sigmoid activation function in the hidden layers. The neural network model can implement dropout technique to prevent overfitting during training. The multi-task machine learning model can be trained using 10-fold cross-validation with an ensemble of 10 models used for final average prediction and standard deviation calculation.

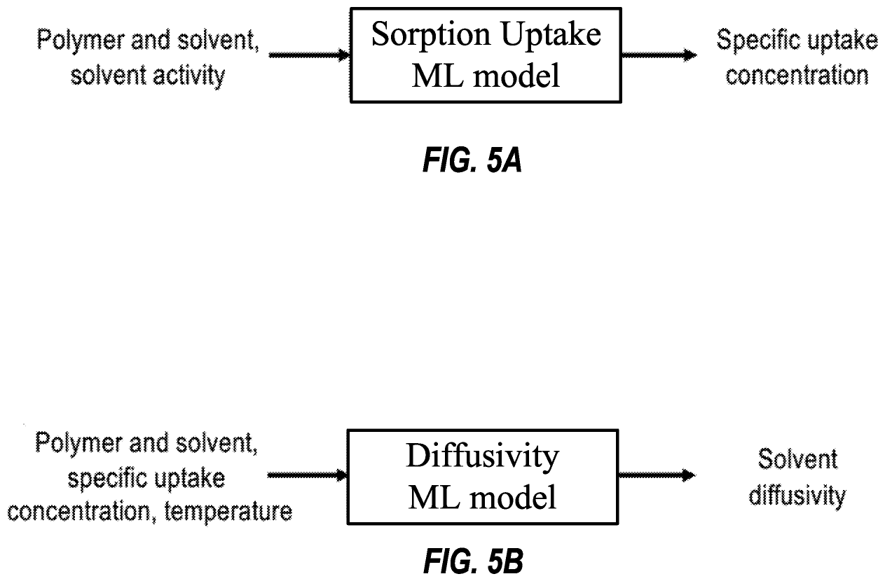

Referring to FIG. 5A, a block diagram illustrates a sorption uptake machine learning model system. The sorption uptake machine learning model system receives inputs comprising polymer and solvent information along with solvent activity data. The sorption uptake machine learning model processes these inputs to generate an output of specific uptake concentration. The sorption uptake machine learning model predicts the amount of solvent absorbed per mass of the polymer matrix based on polymer-solvent fingerprints and solvent activity values provided as inputs.

The sorption uptake machine learning model predicts specific uptake expressed as mmol of solvent present per gram of polymer across a range of activities. The sorption uptake machine learning model comprises an input layer, two hidden layers, and an output layer trained on 2275 systems of 46 polymers and 91 solvents. The sorption uptake machine learning model can be referred to herein as generating solubility coefficient predictions using a sorption uptake machine learning model.

With continued reference to FIG. 5A, the solubility coefficient S can be calculated by normalizing the ML-predicted specific uptake solvent concentration by the solvent saturated vapor pressure. The solubility coefficient can be expressed according to the following equation:

where C represents the solvent concentration predicted by the sorption uptake machine learning model and pₛₐₜ represents the solvent saturated vapor pressure obtained from a chemical database such as PubChem. Polymer densities can be determined using a separate machine learning model for calculating solubility in terms of volume. The solubility coefficient predictions generated by the sorption uptake machine learning model enable calculation of permeability values for polymer candidates.

Referring to FIG. 5B, a block diagram illustrates a diffusivity machine learning model system. The diffusivity machine learning model system receives inputs comprising polymer and solvent information, specific uptake concentration, and temperature data. The diffusivity machine learning model processes these inputs to generate an output of solvent diffusivity. The diffusivity machine learning model predicts the rate at which solvent molecules diffuse through the polymer membrane based on the provided polymer-solvent characteristics, concentration levels, and temperature conditions. The diffusivity machine learning model can comprise the multi-task and physics-enforced learning model 115 described with reference to FIG. 1. Predicting solvent diffusivity values for a plurality of polymers using the trained physics-enforced neural network model enables subsequent calculation of permeability and permselectivity metrics.

As further shown in FIGS. 5A-5B, the sorption uptake machine learning model and the diffusivity machine learning model together enable estimation of solvent permeability through a polymer membrane according to the solution-diffusion mechanism. Calculating permeability values comprises generating solubility coefficient predictions using the sorption uptake machine learning model and calculating permeability as a product of the diffusivity predictions and the solubility coefficient predictions. The permeability P can be calculated according to the following equation:

where D represents the diffusivity predicted by the diffusivity machine learning model and S represents the solubility coefficient predicted by the sorption uptake machine learning model. The instructions stored in the memory can cause the processor to calculate permeability for each polymer candidate as a product of the diffusivity predictions and solubility coefficient predictions generated by the sorption uptake machine learning model.

Calculating permeability values and permselectivity values for the plurality of polymer candidates based on the diffusivity predictions enables identification of polymer membrane candidates. The ideal permselectivity α_{AB} can be calculated according to the following equation:

where P_{A} and P_{B} represent permeabilities, D_{A} and D_{B} represent diffusivities, and S_{A} and S_{B} represent solubility coefficients for pure solvents A and B respectively. Determining permeability and permselectivity metrics for the plurality of polymers based on the predicted solvent diffusivity values enables screening of polymer candidates for membrane applications.

The system can compute permeability and ideal permselectivity for each polymer candidate in the set based on the diffusivity predictions. The system can output polymer membrane candidates having permeability and ideal permselectivity values exceeding predetermined thresholds. Identifying one or more polymer membrane candidates from the plurality of polymer candidates based on the calculated permeability values and permselectivity values enables selection of polymers with optimized separation performance for binary solvent separation applications.

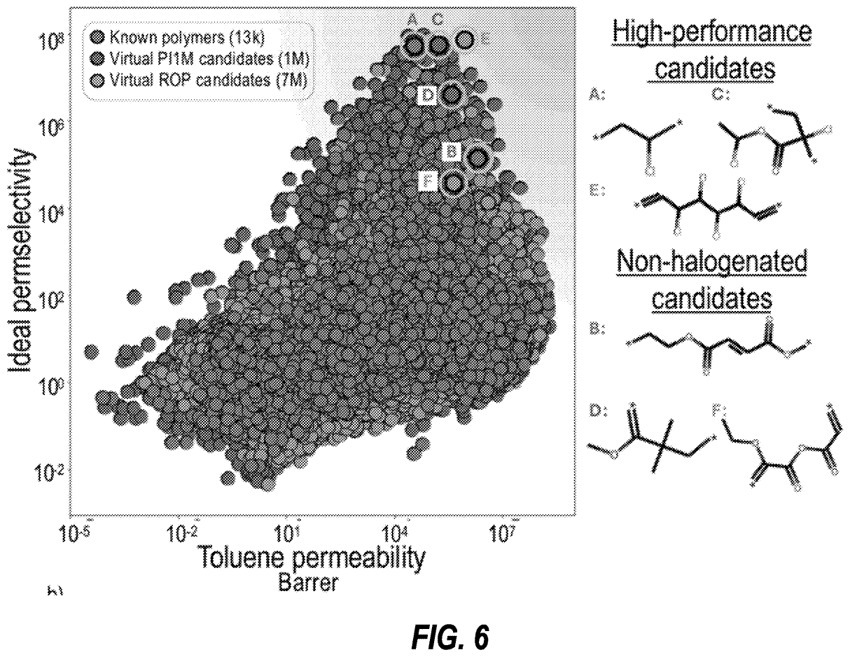

Referring to FIG. 6, a graph illustrates a trade-off plot for polymer membrane candidates in toluene-heptane separation applications. The vertical axis represents ideal permselectivity on a logarithmic scale ranging from 10⁻² to 10⁸. The horizontal axis represents toluene permeability in Barrer units on a logarithmic scale ranging from 10⁻⁵ to 10⁷. The trade-off plot enables visualization of the relationship between permeability and permselectivity for polymer membrane candidates, facilitating identification of polymers with optimized separation performance.

The graph displays three categories of data points representing different polymer candidate sources. A first category comprises known polymers from a polymer database, with the known polymer space comprising approximately 13,000 polymers for screening membrane candidates. A second category comprises virtual PI1M candidates from a PI1M database containing approximately 1 million virtually generated polymers. The PI1M database can be produced using a generative Recurrent Neural Network (RNN) model trained on SMILES strings of existing polymers. A third category comprises virtual ROP candidates from a ring-opening polymerization database comprising approximately 7 million chemically recyclable polymer candidates. The ring-opening polymerization database can be generated using Virtual Forward Synthesis (VFS) that utilizes 30,272,000 known commercial molecules. The plurality of polymer candidates comprises virtually generated polymers from the ring-opening polymerization database as described above.

With continued reference to FIG. 6, the set of polymer candidates comprises at least one of known polymers from the polymer database or virtually generated polymers from the ring-opening polymerization database comprising chemically recyclable polymer candidates. Virtual polymers containing inorganic elements such as Na, P, and Si can be excluded from screening to ensure reliable predictions from the multi-task and physics-enforced learning model 115.

The data points form a distribution pattern across the plot area, with high-performance candidates appearing in an upper right region of the graph where both permeability and ideal permselectivity values are elevated. Identifying one or more polymer membrane candidates comprises applying screening criteria based on a permselectivity threshold and a permeability threshold. The screening criteria can include a moderately stringent criteria of ideal permselectivity greater than 10^{5.5} and permeability greater than 10⁴ Barrer. The screening criteria can include a relaxed criteria of ideal permselectivity greater than 10⁴ while maintaining the permeability threshold of 10⁴ Barrer. Selecting polymer candidates that satisfy both the permselectivity threshold and the permeability threshold enables identification of polymer membrane candidates with optimized separation performance.

As further shown in FIG. 6, six specific polymer candidates are highlighted and labeled as A, C, E, B, D, and F. The right side of the figure displays chemical structures for these highlighted candidates, divided into two groups. High-performance candidates include structures A, C, and E, which contain halogenated functional groups as indicated by chlorine atoms in their molecular structures. Polyvinyl chloride (PVC) can be identified as a benchmark polymer for toluene-heptane separation with predicted toluene permeability of 10^{3.56} Barrer and ideal permselectivity of 10^{7.7}. The identification of PVC as a benchmark polymer validates the methodology of the multi-task and physics-enforced learning model 115 for predicting membrane performance.

Non-halogenated candidates include structures B, D, and F, which represent environmentally friendly alternatives lacking halogen atoms in their molecular frameworks. Identifying one or more polymer membrane candidates further comprises filtering the selected polymer candidates to identify non-halogenated polymer candidates. The instructions stored in the memory can cause the processor to filter the polymer membrane candidates to identify non-halogenated polymer candidates as environmentally friendly alternatives. Identifying non-halogenated polymer candidates from the plurality of polymers that satisfy target permeability and permselectivity criteria for a binary solvent separation application enables discovery of sustainable alternatives to halogenated polymers such as PVC for toluene-heptane separation.

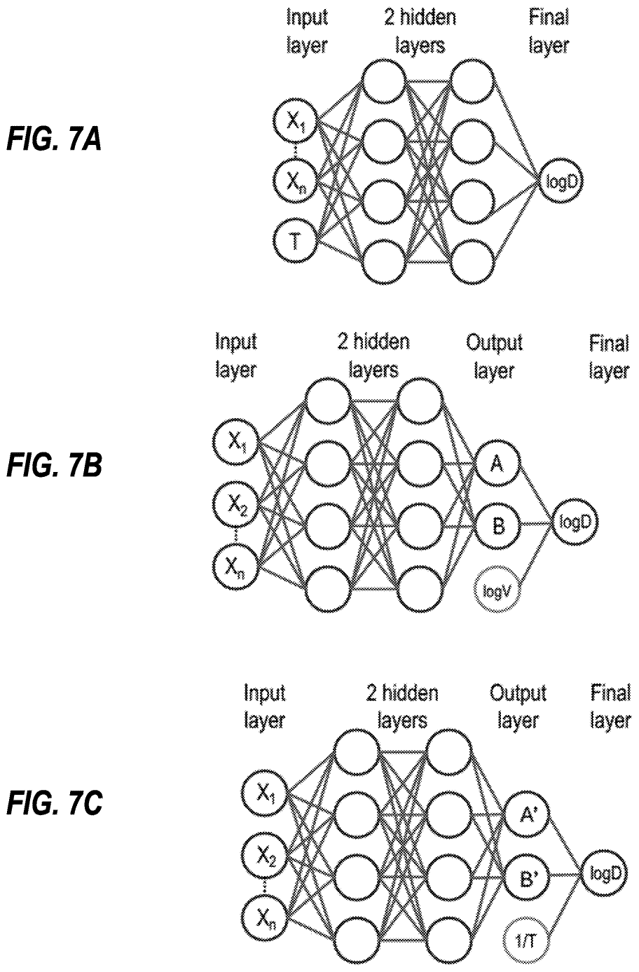

Referring to FIGS. 7A-7C, three neural network architectures for predicting solvent diffusivity in polymers are illustrated. Each architecture comprises an input layer, two hidden layers, and an output layer, with variations in how physical laws are incorporated into the model structure. The multi-task machine learning model can comprise a neural network architecture having an input layer, two hidden layers, and an output layer that incorporates a physics-based relationship.

FIG. 7A depicts a standard neural network architecture. The input layer receives polymer and solvent features designated as X₁ through Xₙ, along with temperature T. These inputs connect to two hidden layers, each containing multiple nodes arranged in a fully connected configuration. The hidden layers process the input features through learned weights and activation functions. The final layer produces a single output representing the logarithm of diffusivity (logD). The standard neural network architecture serves as a baseline model without physics-based constraints for comparison against physics-enforced architectures.

With continued reference to FIGS. 7A-7C, FIG. 7B illustrates a physics-enforced neural network (PENN-1) incorporating an empirical solvent volume law. The input layer accepts features X₁ through Xₙ, which feed into two hidden layers similar to the standard architecture shown in FIG. 7A. The output layer includes three nodes designated as A, B, and logV. The parameters A and B are learned by the network as functions of polymer-solvent chemistry, while logV represents the logarithm of solvent molar volume. The physics-based relationship can comprise an empirical power law relationship between solvent molar volume and diffusivity. The physics-enforced neural network (PENN-1) encodes an empirical power law relationship where diffusivity decreases with increasing solvent molar volume according to the following equation:

where V represents solvent molar volume and A and B are learned parameters. These intermediate outputs are combined in the final layer to produce logD according to the power law relationship between diffusivity and solvent molar volume, enabling the model to capture the slower diffusion behavior of bulkier molecules.

As further shown in FIGS. 7A-7C, FIG. 7C presents a physics-enforced neural network (PENN-2) based on Arrhenius temperature dependence. The input layer receives features X₁ through Xₙ, which connect to two hidden layers. The output layer contains three nodes designated as A′, B′, and 1/T, where 1/T represents the inverse of temperature. The physics-based relationship can comprise an Arrhenius-based temperature dependence relationship that models solvent diffusivity as a function of temperature. The Arrhenius-based temperature dependence relationship encodes a pre-exponential diffusion coefficient and an activation energy as learned parameters of the multi-task machine learning model. The physics-enforced constraint can comprise an Arrhenius-based relationship that encodes temperature dependence of solvent diffusivity through learned parameters representing a pre-exponential diffusion coefficient and an activation energy. The Arrhenius-based relationship can be expressed according to the following equation:

where D₀ represents the pre-exponential diffusion coefficient, E represents the activation energy, R represents the universal gas constant, and T represents temperature. The parameters A′ and B′ correspond to the pre-exponential diffusion coefficient and activation energy terms in the Arrhenius equation, respectively. The final layer combines these outputs to produce logD, enabling the model to extrapolate diffusivity predictions to temperatures outside the training data range.

A non-transitory computer-readable medium can store instructions that, when executed by a processor, cause the processor to perform operations comprising training a physics-enforced neural network model using the training dataset. The physics-enforced neural network model incorporates an Arrhenius-based temperature dependence relationship for solvent diffusivity as described above with reference to FIG. 7C. The physics-enforced neural network architectures shown in FIGS. 7B-7C enable more accurate predictions when extrapolating beyond training data by encoding physical laws governing solvent transport into the model architecture.

The disclosed technology can be further understood according to the following clauses:
Clause 1: A method for identifying polymer membranes for solvent separation, comprising: receiving experimental diffusivity data comprising measurements of solvent diffusivity in polymers; receiving simulated diffusivity data comprising computationally generated solvent diffusivity values for polymer-solvent systems; training a multi-task machine learning model using both the experimental diffusivity data and the simulated diffusivity data, wherein the multi-task machine learning model learns correlations between the experimental diffusivity data and the simulated diffusivity data; encoding a physics-based relationship into the multi-task machine learning model, wherein the physics-based relationship constrains predictions of the multi-task machine learning model according to a physical law governing solvent transport; generating, using the trained multi-task machine learning model, diffusivity predictions for a plurality of polymer candidates; calculating permeability values and permselectivity values for the plurality of polymer candidates based on the diffusivity predictions; and identifying one or more polymer membrane candidates from the plurality of polymer candidates based on the calculated permeability values and permselectivity values.
Clause 2: The method of clause 1, wherein the physics-based relationship comprises an Arrhenius-based temperature dependence relationship that models solvent diffusivity as a function of temperature.
Clause 3: The method of clause 2, wherein the Arrhenius-based temperature dependence relationship encodes a pre-exponential diffusion coefficient and an activation energy as learned parameters of the multi-task machine learning model.
Clause 4: The method of clause 1, wherein the physics-based relationship comprises an empirical power law relationship between solvent molar volume and diffusivity.
Clause 5: The method of clause 1, wherein training the multi-task machine learning model comprises: converting experimental activity-dependent diffusivity data to concentration-dependent diffusivity data using a sorption uptake machine learning model; and combining the converted concentration-dependent diffusivity data with the simulated diffusivity data for training.
Clause 6: The method of clause 1, wherein the simulated diffusivity data is generated using molecular dynamics simulations comprising: generating polymer-solvent structures using a polymer structure predictor; performing an equilibration procedure on the polymer-solvent structures; executing a production run in NPT and NVT ensembles; and calculating diffusivity values from mean squared displacement analysis.
Clause 7: The method of clause 1, wherein calculating permeability values comprises: generating solubility coefficient predictions using a sorption uptake machine learning model; and calculating permeability as a product of the diffusivity predictions and the solubility coefficient predictions.
Clause 8: The method of clause 1, wherein the plurality of polymer candidates comprises virtually generated polymers from a ring-opening polymerization database.
Clause 9: The method of clause 1, wherein identifying one or more polymer membrane candidates comprises: applying screening criteria based on a permselectivity threshold and a permeability threshold; and selecting polymer candidates that satisfy both the permselectivity threshold and the permeability threshold.
Clause 10: The method of clause 9, wherein identifying one or more polymer membrane candidates further comprises filtering the selected polymer candidates to identify non-halogenated polymer candidates.
Clause 11: The method of clause 1, wherein the multi-task machine learning model comprises a neural network architecture having an input layer, two hidden layers, and an output layer that incorporates the physics-based relationship.
Clause 12: A system for polymer membrane design for solvent separation, comprising: a processor; and a memory storing instructions that, when executed by the processor, cause the processor to: receive a combined dataset comprising experimental diffusivity data and simulated diffusivity data for polymer-solvent systems; train a multi-task machine learning model on the combined dataset, wherein the multi-task machine learning model comprises a neural network architecture configured to learn from both the experimental diffusivity data and the simulated diffusivity data; incorporate a physics-enforced constraint into the multi-task machine learning model, wherein the physics-enforced constraint encodes a relationship between solvent diffusivity and at least one of solvent molar volume or temperature; generate diffusivity predictions for a set of polymer candidates using the trained multi-task machine learning model; compute permeability and ideal permselectivity for each polymer candidate in the set based on the diffusivity predictions; and output polymer membrane candidates having permeability and ideal permselectivity values exceeding predetermined thresholds.
Clause 13: The system of clause 12, wherein the physics-enforced constraint comprises an Arrhenius-based relationship that encodes temperature dependence of solvent diffusivity through learned parameters representing a pre-exponential diffusion coefficient and an activation energy.
Clause 14: The system of clause 12, wherein the instructions further cause the processor to: convert experimental activity-dependent diffusivity data to concentration-dependent diffusivity data using a sorption uptake machine learning model; and combine the converted concentration-dependent diffusivity data with the simulated diffusivity data to form the combined dataset.
Clause 15: The system of clause 14, wherein the instructions further cause the processor to calculate permeability for each polymer candidate as a product of the diffusivity predictions and solubility coefficient predictions generated by the sorption uptake machine learning model.
Clause 16: The system of clause 12, wherein the set of polymer candidates comprises at least one of known polymers from a polymer database or virtually generated polymers from a ring-opening polymerization database comprising chemically recyclable polymer candidates.
Clause 17: The system of clause 12, wherein the instructions further cause the processor to filter the polymer membrane candidates to identify non-halogenated polymer candidates as environmentally friendly alternatives.
Clause 18: A non-transitory computer-readable medium storing instructions that, when executed by a processor, cause the processor to perform operations comprising: combining experimental diffusivity data with simulated diffusivity data to form a training dataset for polymer-solvent systems; training a physics-enforced neural network model using the training dataset, wherein the physics-enforced neural network model incorporates an Arrhenius-based temperature dependence relationship for solvent diffusivity; predicting solvent diffusivity values for a plurality of polymers using the trained physics-enforced neural network model; determining permeability and permselectivity metrics for the plurality of polymers based on the predicted solvent diffusivity values; and identifying non-halogenated polymer candidates from the plurality of polymers that satisfy target permeability and permselectivity criteria for a binary solvent separation application.
Clause 19: The non-transitory computer-readable medium of clause 18, wherein the operations further comprise: generating simulated diffusivity data using molecular dynamics simulations that calculate diffusivity values from mean squared displacement analysis of polymer-solvent structures.
Clause 20: The non-transitory computer-readable medium of clause 19, wherein the molecular dynamics simulations comprise: generating polymer-solvent structures using a polymer structure predictor; performing a multi-step equilibration procedure on the polymer-solvent structures; and executing a production run comprising an NPT ensemble phase followed by an NVT ensemble phase.

Machine readable storage including machine-readable instructions, when executed, to implement a method or realize an apparatus in any of the examples of the present application.

Various techniques, or certain aspects or portions thereof, may take the form of program code (i.e., instructions) embodied in tangible media, such as floppy diskettes, CD-ROMs, hard drives, a non-transitory computer readable storage medium, or any other machine-readable storage medium wherein, when the program code is loaded into and executed by a machine, such as a computer, the machine becomes an apparatus for practicing the various techniques. In the case of program code execution on programmable computers, the computing device may include a processor, a storage medium readable by the processor (including volatile and non-volatile memory and/or storage elements), at least one input device, and at least one output device. The volatile and non-volatile memory and/or storage elements may be a RAM, an EPROM, a flash drive, an optical drive, a magnetic hard drive, or another medium for storing electronic data. The eNB (or other base station) and UE (or other mobile station) may also include a transceiver component, a counter component, a processing component, and/or a clock component or timer component. One or more programs that may implement or utilize the various techniques described herein may use an application programming interface (API), reusable controls, and the like. Such programs may be implemented in a high-level procedural or an object-oriented programming language to communicate with a computer system. However, the program(s) may be implemented in assembly or machine language, if desired. In any case, the language may be a compiled or an interpreted language, and combined with hardware implementations.

It should be understood that many of the functional units described in this specification may be implemented as one or more components, which is a term used to more particularly emphasize their implementation independence. For example, a component may be implemented as a hardware circuit comprising custom very large scale integration (VLSI) circuits or gate arrays, off-the-shelf semiconductors such as logic chips, transistors, or other discrete components. A component may also be implemented in programmable hardware devices such as field programmable gate arrays, programmable array logic, programmable logic devices, or the like.

Components may also be implemented in software for execution by various types of processors. An identified component of executable code may, for instance, comprise one or more physical or logical blocks of computer instructions, which may, for instance, be organized as an object, a procedure, or a function. Nevertheless, the executables of an identified component need not be physically located together, but may comprise disparate instructions stored in different locations that, when joined logically together, comprise the component and achieve the stated purpose for the component.

Indeed, a component of executable code may be a single instruction, or many instructions, and may even be distributed over several different code segments, among different programs, and across several memory devices. Similarly, operational data may be identified and illustrated herein within components, and may be embodied in any suitable form and organized within any suitable type of data structure. The operational data may be collected as a single data set, or may be distributed over different locations including over different storage devices, and may exist, at least partially, merely as electronic signals on a system or network. The components may be passive or active, including agents operable to perform desired functions.

Reference throughout this specification to “an example” means that a particular feature, structure, or characteristic described in connection with the example is included in at least one embodiment of the present invention. Thus, appearances of the phrase “in an example” in various places throughout this specification are not necessarily all referring to the same embodiment.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on its presentation in a common group without indications to the contrary. In addition, various embodiments and examples of the present invention may be referred to herein along with alternatives for the various components thereof. It is understood that such embodiments, examples, and alternatives are not to be construed as de facto equivalents of one another, but are to be considered as separate and autonomous representations of the present invention.

Although the foregoing has been described in some detail for purposes of clarity, it will be apparent that certain changes and modifications may be made without departing from the principles thereof. It should be noted that there are many alternative ways of implementing both the processes and apparatuses described herein. Accordingly, the present embodiments are to be considered illustrative and not restrictive, and the invention is not to be limited to the details given herein, but may be modified within the scope and equivalents of the appended claims.

Those having skill in the art will appreciate that many changes may be made to the details of the above-described embodiments without departing from the underlying principles of the invention. The scope of the present invention should, therefore, be determined only by the following claims.

1. A method for identifying polymer membranes for solvent separation, comprising:
receiving experimental diffusivity data comprising measurements of solvent diffusivity in polymers;
receiving simulated diffusivity data comprising computationally generated solvent diffusivity values for polymer-solvent systems;
training a multi-task machine learning model using both the experimental diffusivity data and the simulated diffusivity data, wherein the multi-task machine learning model learns correlations between the experimental diffusivity data and the simulated diffusivity data;
encoding a physics-based relationship into the multi-task machine learning model, wherein the physics-based relationship constrains predictions of the multi-task machine learning model according to a physical law governing solvent transport;
generating, using the trained multi-task machine learning model, diffusivity predictions for a plurality of polymer candidates;
calculating permeability values and permselectivity values for the plurality of polymer candidates based on the diffusivity predictions; and
identifying one or more polymer membrane candidates from the plurality of polymer candidates based on the calculated permeability values and permselectivity values.

2. The method of claim 1, wherein the physics-based relationship comprises an Arrhenius-based temperature dependence relationship that models solvent diffusivity as a function of temperature.

3. The method of claim 2, wherein the Arrhenius-based temperature dependence relationship encodes a pre-exponential diffusion coefficient and an activation energy as learned parameters of the multi-task machine learning model.

4. The method of claim 1, wherein the physics-based relationship comprises an empirical power law relationship between solvent molar volume and diffusivity.

5. The method of claim 1, wherein training the multi-task machine learning model comprises:
converting experimental activity-dependent diffusivity data to concentration-dependent diffusivity data using a sorption uptake machine learning model; and
combining the converted concentration-dependent diffusivity data with the simulated diffusivity data for training.

6. The method of claim 1, wherein the simulated diffusivity data is generated using molecular dynamics simulations comprising:
generating polymer-solvent structures using a polymer structure predictor;
performing an equilibration procedure on the polymer-solvent structures;
executing a production run in NPT and NVT ensembles; and
calculating diffusivity values from mean squared displacement analysis.

7. The method of claim 1, wherein calculating permeability values comprises:
generating solubility coefficient predictions using a sorption uptake machine learning model; and
calculating permeability as a product of the diffusivity predictions and the solubility coefficient predictions.

8. The method of claim 1, wherein the plurality of polymer candidates comprises virtually generated polymers from a ring-opening polymerization database.

9. The method of claim 1, wherein identifying one or more polymer membrane candidates comprises:
applying screening criteria based on a permselectivity threshold and a permeability threshold; and
selecting polymer candidates that satisfy both the permselectivity threshold and the permeability threshold.

10. The method of claim 9, wherein identifying one or more polymer membrane candidates further comprises filtering the selected polymer candidates to identify non-halogenated polymer candidates.

11. The method of claim 1, wherein the multi-task machine learning model comprises a neural network architecture having an input layer, two hidden layers, and an output layer that incorporates the physics-based relationship.

12. A system for polymer membrane design for solvent separation, comprising:
a processor; and
a memory storing instructions that, when executed by the processor, cause the processor to:
receive a combined dataset comprising experimental diffusivity data and simulated diffusivity data for polymer-solvent systems;
train a multi-task machine learning model on the combined dataset, wherein the multi-task machine learning model comprises a neural network architecture configured to learn from both the experimental diffusivity data and the simulated diffusivity data;
incorporate a physics-enforced constraint into the multi-task machine learning model, wherein the physics-enforced constraint encodes a relationship between solvent diffusivity and at least one of solvent molar volume or temperature;
generate diffusivity predictions for a set of polymer candidates using the trained multi-task machine learning model;
compute permeability and ideal permselectivity for each polymer candidate in the set based on the diffusivity predictions; and
output polymer membrane candidates having permeability and ideal permselectivity values exceeding predetermined thresholds.

13. The system of claim 12, wherein the physics-enforced constraint comprises an Arrhenius-based relationship that encodes temperature dependence of solvent diffusivity through learned parameters representing a pre-exponential diffusion coefficient and an activation energy.

14. The system of claim 12, wherein the instructions further cause the processor to:
convert experimental activity-dependent diffusivity data to concentration-dependent diffusivity data using a sorption uptake machine learning model; and
combine the converted concentration-dependent diffusivity data with the simulated diffusivity data to form the combined dataset.

15. The system of claim 14, wherein the instructions further cause the processor to calculate permeability for each polymer candidate as a product of the diffusivity predictions and solubility coefficient predictions generated by the sorption uptake machine learning model.

16. The system of claim 12, wherein the set of polymer candidates comprises at least one of known polymers from a polymer database or virtually generated polymers from a ring-opening polymerization database comprising chemically recyclable polymer candidates.

17. The system of claim 12, wherein the instructions further cause the processor to filter the polymer membrane candidates to identify non-halogenated polymer candidates as environmentally friendly alternatives.

18. A non-transitory computer-readable medium storing instructions that, when executed by a processor, cause the processor to perform operations comprising:
combining experimental diffusivity data with simulated diffusivity data to form a training dataset for polymer-solvent systems;
training a physics-enforced neural network model using the training dataset, wherein the physics-enforced neural network model incorporates an Arrhenius-based temperature dependence relationship for solvent diffusivity;
predicting solvent diffusivity values for a plurality of polymers using the trained physics-enforced neural network model;
determining permeability and permselectivity metrics for the plurality of polymers based on the predicted solvent diffusivity values; and
identifying non-halogenated polymer candidates from the plurality of polymers that satisfy target permeability and permselectivity criteria for a binary solvent separation application.

19. The non-transitory computer-readable medium of claim 18, wherein the operations further comprise:
generating simulated diffusivity data using molecular dynamics simulations that calculate diffusivity values from mean squared displacement analysis of polymer-solvent structures.

20. The non-transitory computer-readable medium of claim 19, wherein the molecular dynamics simulations comprise:
generating polymer-solvent structures using a polymer structure predictor;
performing a multi-step equilibration procedure on the polymer-solvent structures; and
executing a production run comprising an NPT ensemble phase followed by an NVT ensemble phase.
